(19) United States
(12) Patent Application Publication
(10) Pub. No.: US 2008/0294148 A1
(43) Pub. Date: Nov. 27, 2008

(54) SYSTEM AND METHOD FOR REFILLING AN IMPLANTED DELIVERY DEVICE

(76) Inventor: Albert Gardner, Stuart, FL (US)

Correspondence Address: GARDNER GROFF GREENWALD & VILLANUEVA. PC, 2018 POWERS FERRY ROAD, SUITE 800, ATLANTA, GA, 30339, US

(21) Appl. No.: 12/123,593

(22) Filed: May 20, 2008

Related U.S. Application Data
(60) Provisional application No. 60/939,408, filed on May 22, 2007.

(51) Int. Cl. A61K 9/22

(52) U.S. Cl. 604/891.1

(57) ABSTRACT

An apparatus for refilling an implanted delivery device has a needle connected to an output port of a three-way valve. One input port is sealed and the other is connected to a fluid source. When the valve is in a fluid delivery position, fluid from the fluid source flows through the needle, and when the valve is in a stylet insertion position, fluid from the fluid source is prevented from flowing through the needle. When the valve is in the stylet insertion position, a stylet can be inserted through the second input port and into the lumen of the needle, thereby occluding the needle's lumen. In this configuration, a practitioner inserts the needle with stylet therein into the body of the patient to engage the devices's refill port. The stylet is removed, the fluid delivery path is opened, and medication is delivered to the device's reservoir without being contaminated with tissue.

### CROSS REFERENCE TO RELATED APPLICATION

This application claims priority to co-pending U.S. Provisional Patent Application Ser. No. 60/939,408, filed May 22, 2007, which is hereby incorporated herein by reference in its entirety for all purposes.

### TECHNICAL FIELD

The present invention relates generally to the field of surgical instruments and methods, and more particularly to a system and method for refilling an implanted delivery device, such as an intrathecal pump.

### BACKGROUND OF THE INVENTION

Intrathecal drug delivery is a method by which a low dose of a medication, typically a narcotic such as morphine, is delivered to the intrathecal space (i.e., within the spinal canal). A typical intrathecal drug delivery system includes a drug delivery pump implanted in the patient's abdomen, which has a reservoir for the medication, and an intrathecal catheter leading from the pump into the intrathecal space. The pump releases the medication at a set rate, and the medication flows from the pump, through the catheter, and to the site of delivery in the intrathecal space. Intrathecal drug delivery is beneficial because typically smaller doses of the medication can be used to gain relief, as compared to the dosages of those medications taken orally.

In order to maintain a constant supply of medication, a practitioner periodically refills the pump's reservoir by inserting a needle of a syringe containing the appropriate medication into the patient and into a refill port of the pump. The inventor of the present application has discovered that this process tends to cause contamination of the medication with the patient's own blood and tissue. Over time, the intrathecal catheter can clog and its distal ports can become obstructed by rust-like material (derived from the blood's hemoglobin). For example, the prior art catheters depicted in FIG. 1 show such obstructions. The catheter 1 on the left shows obstructions from a catheter that was removed from a patient. The catheter 2 on the right shows obstructions after three days from the start of an experiment simulating conditions leading to obstructions of the catheter. As part of the experiment, a new MEDTRONIC® pump 8637-40 and catheter 8709 were used. The pump was filled with 10 cc of preservative free morphine (HOSPIRA brand) and 0.1 cc of human blood to simulate contamination from pump refills. The pump was programmed to deliver 10 mg of morphine per day with a 0.4 cc per day flow rate. The pump and catheter were placed in a water bath at 98 degrees (which simulates average body temperature of a human being). Within four days, the distal delivery ports of the catheter were becoming obstructed with a black-colored material, as depicted in FIG. 1. Generally, once obstruction of the catheter occurs, the intrathecal catheter must be replaced. When no blockages of the catheter occur, such catheters typically are replaced about every 10 years. However, with the frequency of blockages, catheters are generally replaced about every 1½ to 2 years, thus substantially increasing the number of surgeries and recovery time patients must endure.

Therefore, a need exists for an apparatus and method that allows a practitioner to safely and effectively refill the intrathecal pump reservoir without contaminating the supply of medication contained therein.

### SUMMARY OF THE INVENTION

Advantageously, the present invention provides an apparatus and method that prevents, or at least minimizes, contamination of an implanted delivery device, such as an intrathecal pump, reservoir with blood and tissue, which leads to obstruction of an associated implanted catheter, such as an intrathecal catheter. For example, prevention of obstruction of an intrathecal catheter would prevent numerous re-operations to replace obstructed catheters and would subsequently save hundreds of thousands of health care dollars and save patients the time and pain associated with the multiple re-operations.

In an example form, the present invention is an apparatus for refilling an implanted delivery device. The apparatus includes a needle having a lumen extending therethrough. The proximal end of the needle is connected to an output port of a three-way valve, which regulates the flow of a fluid through the lumen of the needle. A fluid source is connected to a first input port of the valve. When the valve is in a fluid delivery position, fluid from the fluid source flows through the needle, and when the valve is in a stylet insertion position, fluid from the fluid source is prevented from flowing through the needle. Additionally, the apparatus includes a stylet that is inserted through a second input port of the valve and into the lumen of the needle when the valve is in the stylet insertion position. With the stylet substantially occluding the lumen of the needle, a practitioner can refill an implanted medical delivery device (e.g., pump) without contaminating the refill medication with the patient's tissue, which would ordinarily enter the lumen of the needle.

In another form, the present invention comprises a method for refilling a reservoir of a medical pump implanted in a human or animal subject. The method includes the steps of (1) inserting a needle into a human or animal subject, wherein the needle has an inner lumen and wherein a stylet substantially occludes the inner lumen; (2) guiding the needle towards a refill port of the medical pump; (3) engaging the refill port with the needle; (4) removing the stylet from the needle; and (5) injecting a fluid into the medical pump.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 shows photographs of a pair of prior art catheters obstructed with a rust-like material.

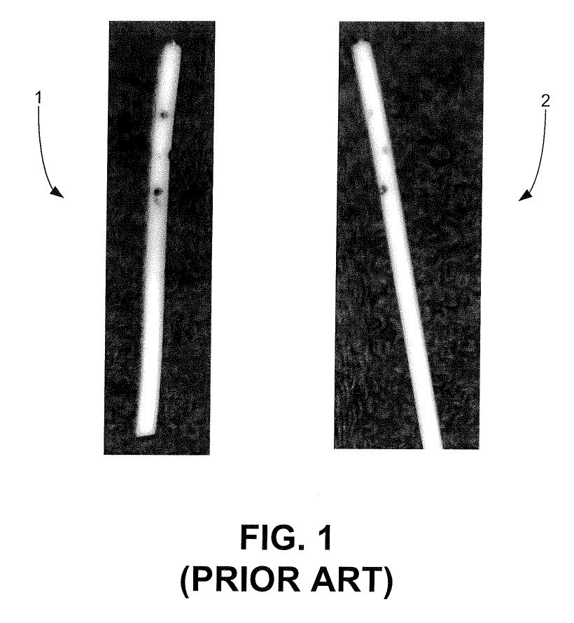

FIG. 2 is a perspective view of an apparatus for refilling an implanted medical delivery device according to an example embodiment of the present invention.

FIG. 3 is a cross-sectional view of a portion of the apparatus of FIG. 2 taken along lines 3-3.

FIG. 4 is a perspective view of an apparatus for refilling an implanted medical delivery device according to another example embodiment of the present invention.

FIG. 5 is a cross-sectional view of a portion of the apparatus of FIG. 2 taken along lines 5-5.

FIG. 6 is a flow diagram of a method of refilling an implanted medical delivery device according to an example embodiment of the present invention.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

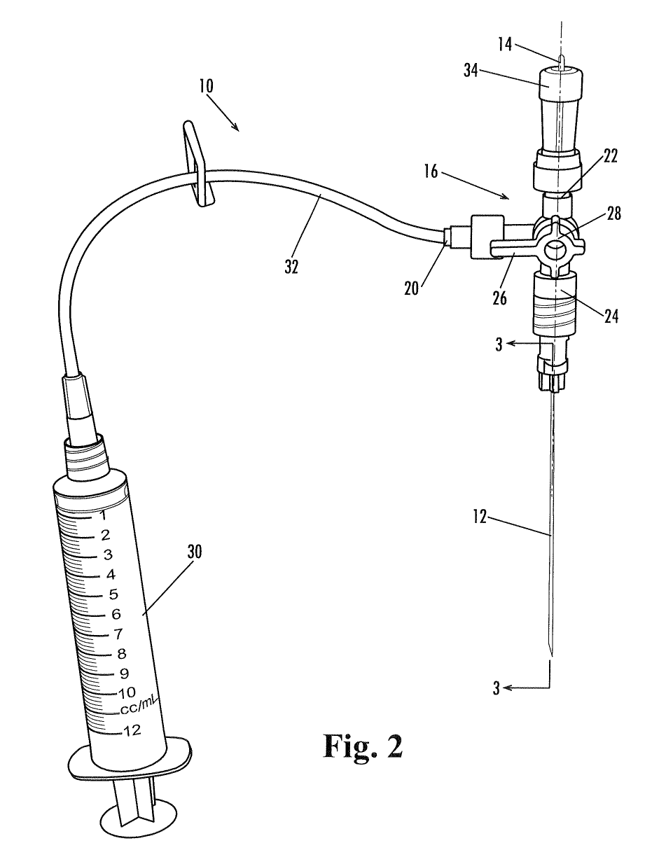

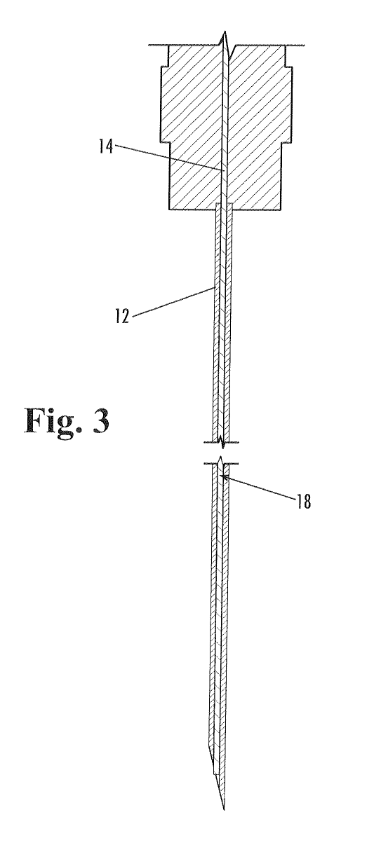

FIG. 2 depicts a refill system 10 for an implanted delivery device (sometimes referred to herein as an “implanted medical pump”) according to an example embodiment of the present invention. The refill system 10 of the present invention can be used to refill implanted medical pumps having a reservoir that holds a fluid, such as a medication. Preferably, the system 10 is used to refill an intrathecal pump, but those skilled in the art will understand that the system of the present invention can be used to refill other implanted delivery devices, including insulin pumps and infusion pumps. The system 10 includes a needle 12 with a stylet 14. Preferably, the needle has a length that is long enough to reach the site of the implanted medical pump from a point external to the body. Additionally, the needle 12 preferably has a gauge sufficient to deliver medication to the refill port of the implanted medical pump. In the case of intrathecal pump refill, the needle 12 is preferably a non-coring intrathecal pump refill needle, such as a 22-gauge, 3½″ long, non-coring spinal needle. The refill needle 12 includes a lumen 18, as shown in sectional view of FIG. 3, for carrying a fluid, such as an opiate, narcotic, other medication, nutrient, hormone solution, or other fluid.

Preferably, the stylet 14 is a high tolerance, flexible stylet that occludes the lumen 18 of the needle 12 while the needle is inserted into the body of a human or animal subject. Alternatively, the stylet 14 can be rigid or substantially rigid. Preferably, the stylet 14 fully occludes the needle's lumen 18 and extends the entire length of the lumen. Thus, the stylet 14 has a size and shape to fully occlude the lumen 18 of the needle 12, while still be configured to slide along the lumen. In other words, the stylet 14 is slidingly received in the lumen 18 of the needle 12 with a snug fit. Moreover, the stylet 14 is preferably longer than the length of the needle so that the proximal end of the stylet can be manipulated by a practitioner. For example, a stylet obtained from a 7″ long, 22-gauge spinal needle can be used with the 22-gauge, 3½″ long non-coring spinal needle for refilling an intrathecal pump. However, those skilled in the art will understand that any suitable gauge and length of needle and stylet can be used and be within the scope of the present invention.

To maintain sterility and to avoid contamination, the needle 12 is connected to a valve 16 to regulate flow through the needle 12. Preferably, the valve 16 is a three-way stopcock having two input ports 20 and 22, an output port 24, and a manually-operated switch 26 for directing flow from one of the two input ports to the output port. Preferably, the output port 24 is aligned or in fluid communication with each of the input ports 20 and 22. Also preferably, the second input port 22 and the output port 24 are axially aligned along longitudinal axis 28. More preferably, the output port 24 is aligned with the opening of the lumen 18 such that the stylet 14 can be removably inserted into the input port 22, through the output port 24, and into the lumen 22. Additionally, the first input port 20 preferably extends transversely relative to the longitudinal axis 28. In other words, the first input port 20 and the output port 24 are nonaxially aligned.

The first input port 20 of the valve 16 is connected to a fluid source, such as a syringe 30. Flexible tubing 32 can be used to connect the tip of the syringe 30 to the first input port 20. The second input port 22 of the valve 16 is sealed with a cap 34. The cap 34 permits the stylet 14 to pass therethrough while simultaneously preventing loss of reservoir fluid (in the medical pump). Preferably, the cap 34 comprises a luer lock latex-free septum as the seal, although those skilled in the art will understand that other suitable seals can be employed as well. The output port 24 of the valve 16 connects to the proximal end of the needle 12 such that the output port 24 and the lumen 18 are in fluid communication.

As described herein, the valve 16 is considered to be in its stylet insertion position when the switch 26 is set such an axial path is opened between the second input port 22 and the output port 24. In the stylet insertion position, a practitioner can insert the stylet 14 through the cap 34 of the second input port 22, through the output port 24, and into the lumen 18 of the needle 12. Notably, fluid cannot pass to/from the fluid source 30 through the lumen 18 of the needle 12. The valve 16 is considered to be its fluid delivery position when the switch 26 is set such that it opens a nonaxial path between the first input port 20 and the output port 24 for fluid delivery through the needle 12. In other words in the fluid delivery position, fluid passes to/from the fluid source 30 through the lumen 18 of the needle 12.

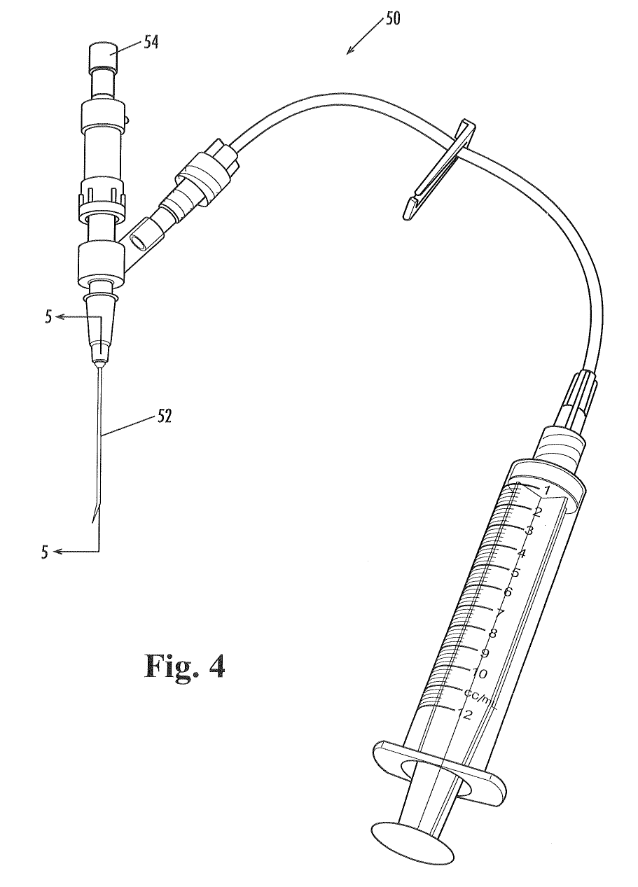

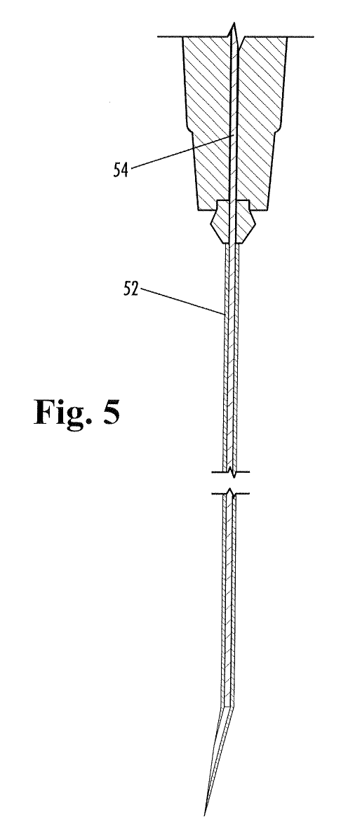

FIGS. 4 and 5 depict an intrathecal pump refill system 50 according to another example embodiment of the present invention. The system 50 is substantially similar to the system 10 but with the exception of the needle 52. Preferably, the needle 52 is a huber needle having a beveled tip.

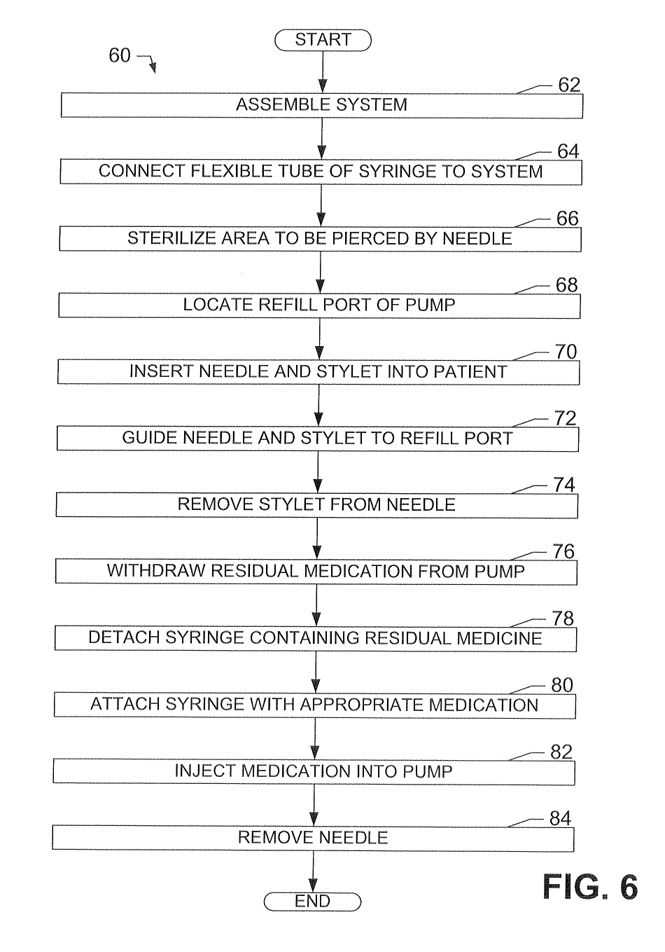

Referring now to FIG. 6, in operation, a practitioner uses one of the systems 10 or 50 of the present invention to refill the medication reservoir of an intrathecal pump implanted in a patient. Although the following method 60 is described in terms of the system 10, the method can also be employed with the system 50. Additionally, the method 60 is described in terms of refilling an intrathecal pump, but those skilled in the art will understand that the method can be easily adapted to refill other implanted delivery devices/pumps. Beginning at step 62, the practitioner assembles the system, if not already done so, by securing the cap 34 to the second input port 22 of the valve 16 and securing the needle 12 to the output port 24 of the valve. The practitioner manipulates the valve switch 26 to its stylet insertion position at step 64, inserts the stylet 14 through the cap 34 and septum, and advances the stylet to the distal tip of the needle 12. Those skilled in the art will further understand that the needle 12, stylet 14, valve 16, and cap 34 can be preassembled and/or sold as a kit.

At step 64, the practitioner connects the remaining input port 20 of the valve 16 to the flexible tubing 32, which is preferably connected to an empty syringe. At step 66, the practitioner prepares the patient for the procedure by sterilizing the area of the patient's body corresponding to the place where the intrathecal pump is implanted and covers it with a sterile drape. Optionally, the practitioner can anesthetize the sterile area. As generally well known, the practitioner uses a plastic template to help locate the refill port of the intrathecal pump at step 68, as the refill port of a conventional intrathecal pump is typically located at the center of the pump. The practitioner inserts the needle 12 with stylet 14 therein into the patient at step 70 and guides it to the intrathecal pump's refill port at step 72. Once the refill port is engaged, the practitioner removes the stylet 14 from the needle 12 at step 74. At step 76, the practitioner manipulates the valve switch 26 to open the nonaxial path of the valve 16 (i.e., the path between the fluid source 30 and the output port 24) and withdraws any residual medication from the pump by pulling back on the plunger of the empty syringe. Once the residual medication is withdrawn, the practitioner manipulates the valve switch 26 to close the nonaxial path of the valve 16 and detaches the syringe holding the residual medication at step 78. Optionally, the practitioner can measure the residual medication, as is generally well known to those skilled in the art. The practitioner then at step 80 attaches a new syringe prefilled with the appropriate type and amount of medication to the second input port 22 of the valve 16 and manipulates the valve switch 26 to the valve's fluid delivery position. At step 82, the practitioner slowly injects the medication into the pump by depressing the plunger of the syringe. At step 84, the practitioner removes the needle 12 from the patient, and the process 60 of refilling the pump ends. As generally well known in the art, the pump is then typically checked and programmed.

Advantageously, the apparatus and method of the present invention minimizes, if not eliminates, the potential for the medication to become contaminated with blood, skin, hair, tissues, etc. Such potential for contamination is greatly minimized because the lumen of the needle is preferably completely occluded as it is being inserted into the refill port of the pump. Thus minimal, or even no, blood or tissue can enter the lumen of the needle. Consequently, medication with minimal contamination will be delivered through the associated catheter (e.g., intrathecal catheter), resulting in fewer blockages of the catheter. Thus, catheters will need to be replaced less frequently.

Those skilled in the art will further understand that a substantially similar needle can be constructed for use with the catheter access port of the pump so as to prevent additional contamination. For example, a 24-gauge or 25-gauge needle can be used with a corresponding stylet (as typically a smaller gauge needle is used with the catheter access port).

While the invention has been described with reference to preferred and example embodiments, it will be understood by those skilled in the art that a variety of modifications, additions and deletions are within the scope of the invention, as defined by the following claims.

What is claimed is:

1. An apparatus for refilling an implanted delivery device in a human or animal subject, comprising:
a needle having a lumen extending therethrough, wherein the needle has a proximal end and a distal end;
a three-way valve for regulating flow of a fluid, wherein an output port of the valve is connected to the proximal end of the needle;
a fluid source, wherein the fluid source is connected to a first input port of the valve, wherein when the valve is in a fluid delivery position, fluid from the fluid source flows through the needle, and wherein when the valve is in a stylet insertion position, fluid from the fluid source is prevented from flowing through the needle; and
a stylet for removable insertion through a second input port of the valve and into the lumen of the needle when the valve is in the stylet insertion position.

2. The apparatus of claim 1, wherein the needle is a huber needle.

3. The apparatus of claim 1, wherein the needle is a non-coring intrathecal needle.

4. The apparatus of claim 1, wherein the valve comprises a stopcock.

5. The apparatus of claim 1, wherein the third port of the stopcock is sealed with a material that permits the stylet to be inserted but prohibits flow of a fluid therethrough.

6. The apparatus of claim 5, wherein the material is a latex-free material.

7. The apparatus of claim 1, wherein the stylet substantially occludes the lumen of the needle when installed.

8. The apparatus of claim 1, wherein the fluid source is a syringe.

9. The apparatus of claim 1, further comprising a switch for manipulating the valve from the stylet insertion position to the fluid delivery position.

10. A method for refilling a reservoir of a medical pump implanted in a human or animal subject, comprising:
inserting a needle into a human or animal subject, wherein the needle has an inner lumen and wherein a stylet substantially occludes the inner lumen;
guiding the needle towards a refill port of the medical pump;
engaging the refill port with the needle;
removing the stylet from the needle; and
injecting a fluid into the medical pump.

11. The method of claim 10, further comprising removing residual medication from the medical pump prior to injecting the fluid into the pump.

12. The method of claim 10, wherein the needle is connected to a valve that regulates flow of a fluid therethrough.

13. The method of claim 12, wherein the valve is a three-way valve.

14. A kit for refilling an implantable medical pump, comprising:
a needle;
a stylet;
a three-way stopcock having two input ports and one output port, wherein one of the input ports is sealed; and
a syringe, wherein all of the above are packaged together in a single kit.

15. The kit of claim 14, wherein the needle is an intrathecal needle.

16. An apparatus for fluid delivery to a reservoir, said apparatus comprising:
a valve operable between a first position and a second position, wherein the first position allows communication between a first port and a second port, and wherein the second position allows communication between the first port and a third port;
a needle in communication with the first port, said needle having a lumen extending therethrough;
a fluid source in communication with the second port; and
a stylet insertable through the third port and extending through the lumen of the needle.

17. The apparatus of claim 16, wherein the stylet substantially occludes the lumen of the needle when installed.
